(19) United States
(12) Patent Application Publication
(10) Pub. No.: US 2023/0194522 A1
(43) Pub. Date: Jun. 22, 2023

(54) METHODS AND KITS FOR ASSAYING A LARGE FLUID VOLUME USING FLOW CYTOMETRY

(71) Applicant: HACH COMPANY, Loveland, CO (US)

(72) Inventor: Dan KROLL, Fort Collins, CO (US)

(73) Assignee: HACH COMPANY, Loveland, CO (US)

(21) Appl. No.: 18/083,656

(22) Filed: Dec. 19, 2022

Related U.S. Application Data
(60) Provisional application No. 63/291,796, filed on Dec. 20, 2021.

(51) Int. Cl. G01N 33/569; G01N 33/543; G01N 15/14; G01N 15/06

(52) U.S. Cl. CPC G01N 33/56911; G01N 33/54326; G01N 15/1404; G01N 15/06; G01N 2015/149; G01N 2015/1493; G01N 2015/0693; G01N 2015/1006

(57) ABSTRACT

Methods and kits are provided for analyzing a fluid with a flow cytometer to determine the concentration of a target component in the fluid. At least two bead groups are combined with the fluid, wherein each of the bead groups includes surface-functionalized beads that have a different size from the other bead groups. The target component can bind to the functional groups on the beads, and the beads with the targets can be counted with the flow cytometer. Based on the numbers of beads with targets in each group, a most probable number (MPN) of the target component can be determined.

### CROSS-REFERENCE TO RELATED APPLICATIONS

This application claims the filing date benefit of U.S. Provisional Application No. 63/291,796, which was field on Dec. 20, 2021. The entirety of this prior application is incorporated by reference herein.

### BACKGROUND

Flow cytometry is a technique that is used to measure physical and/or chemical characteristics of a population of cells or particles. In a typical flow cytometry process, a fluid sample containing cells or particles is injected into a flow cytometer instrument. The cells or particles may be labeled with fluorescent markers. For example, the markers can include vital dyes that can differentiate live cells from dead cells. The flow cytometer instrument includes a laser beam that directs light through the fluid sample and detectors measure light scattering and fluorescence or colorimetric properties. The process and instrument are ideally configured so that cells and particles pass single-file through the laser beam, i.e., the cells or particles are monodispersed. Accordingly, the cells or particles can be counted, and sorted and/or further characterized based on the light scattering and/or fluorescent/colorimetric properties that are detected.

Flow cytometry is a useful tool that can provide accurate counts of various types of organisms in liquid samples, such as water. One drawback of flow cytometry technology is that only a very small column of water can be interrogated. For example, high flow rate flow cytometers can only interrogate between about 100 and 1000 μL/min. Thus, for many fluid sample volumes, the time required to complete the assay can be a hindrance, And when small samples sizes are measured, extrapolation of measured data to larger sample values can be unreliable where the sample has a low concentration of the target of interest (e.g., microorganism).

In many cases where large sample volumes are desired or required, the assay time required for flow cytometry is considered unacceptable. For example, a sample volume of 100 mL will take about 1 hour and 40 minutes to analyze when using a flow cytometer with a 1000 μL/min capacity. In many applications, the sample throughput of flow cytometry is not adequate. For example, when analyzing organisms in water it is often desirable or required to interrogate water volumes of 100 mL or more, and even up to hundreds of liters, so that the sample size is considered sufficiently representative.

To address these issues, various means of sample concentration have been developed, including filtration, centrifugation, and immunomagnetic separation. Although these techniques decrease the sample volume that needs to be passed through the flow cytometer, they can be cumbersome and expensive, particularly if applied to concentrate very large sample volumes. Some of these techniques may also compromise cell viability, which hinders an accurate live/dead count.

### SUMMARY

Accordingly, in some aspects, this disclosure provides rapid methods and kits to reliably interrogate large volumes. In the context of microbiological assays, the methods can also discriminate live/dead populations.

In one aspect, this disclosure provides a method of analyzing a fluid sample to determine the amount of a target component in the fluid sample. The method includes combining a first group of beads with the fluid sample and combining a second group of beads with the fluid sample. The first group of beads has beads with a first size that include a surface functional group and/or moiety that can bind to the target component, and the second group of beads has beads with a second size that is different from the first size which also includes the surface functional group and/or moiety that can bind to the target component. The target component is allowed to bind the surface functional group and/or moiety of the first and second groups of beads. The method also includes labeling the target component with a fluorescence marker, and separating the first and second group of beads from the fluid sample and then analyzing the separated first and second group of beads with a flow cytometer to determine (i) a number of beads in the first group of beads that includes the target component; and (ii) a number of beads in the second group of beads that includes the target component. The method can also include determining the most probable number (MPN) of the target component in the fluid sample based on number of beads in each group that have the target component.

In another aspect, this disclosure provides a method of analyzing a fluid that includes a target component. The method includes combining at least two bead groups with the fluid, where each of the bead groups includes a plurality of surface-functionalized beads that have a different size from beads in each of the other bead groups, and the target component in the fluid binds to at least some of the plurality of surface-functionalized beads. The method includes binding a fluorescent or colorimetric marker to the target component, and then introducing the beads of the at least two bead groups into a flow cytometer, and detecting, with the flow cytometer, a size of each of the beads and fluorescence/colorimetric properties of each of the beads.

In another aspect, this disclosure provides a kit that includes a first group of magnetic beads having a first size, a second group of magnetic beads having a second size that is different from the first size, and a fluorescence marker. The first and second groups of magnetic beads are surface-functionalized with the same functional group or moiety.

### BRIEF DESCRIPTION OF THE DRAWINGS

FIG. 1 is a schematic diagram illustrating a method of assaying a water sample according to one embodiment; and

FIG. 2 is a schematic diagram of a flow cytometer.

### DETAILED DESCRIPTION OF EMBODIMENTS

This disclosure provides methods, systems, and kits for analyzing a liquid sample with a flow cytometer by functionally subdividing the sample into two or more groups, and detecting characteristics of target(s) of interest in each group using a flow cytometer. More specifically, and as explained in detail below, aspects of this disclosure functionally subdivide the liquid sample into groups by using two or more sizes of functionalized beads that capture or bind to the target(s) in the sample. The functionalized beads are injected into the flow cytometer, which can count the number of beads of each size group that are bound to targets. The overall number of targets in the fluid sample can be extrapolated using a statistical method, such as a most probable numbers method.

By way of background, a most probable numbers (MPN) analysis (also known as the method of Poisson zeroes) has been used to estimate bacterial populations in samples where the sample is subdivided into multiple degrees of dilution (e.g., 10× or 2×) and assessing the presence or absence of the bacterial population in each degree of dilution. The degree of dilution at which the absence of the organism begins to show indicates that the sample has been diluted so much that there are many subsamples in which none appear. A suite of replicates at any given concentration allows finer resolution so that the number of positive and negative samples can be used to estimate the original concentration within an appropriate order of magnitude. In microbiology, samples have been assessed by this method by incubating cultures and assessing and counting the presence or absence in a subsample by eye. An alternative to this serial-dilution-to-extinction method involves dividing a sample into several discrete compartments of various sizes without performing a dilution. For example, this can be done with a Quanti-Tray system (Indexx), in which a liquid sample is divided into a number of different sized compartments, and each compartment is evaluated for a negative or positive test. The statistical likelihood of an organism landing in a given compartment allows a MPN calculation as to the total organism numbers within a range. Thus, the extrapolated/estimated count in the overall sample can be determined using a statistical table or a statistical calculation based on the number of positives in each compartment size group.

The methods, systems, and kits described herein allow for a similar MPN extrapolation in a flow cytometry application by effectively subdividing the sample with different sized functionalized beads. Conceptually, the functionalized beads of at least two sizes take the place of the different sized compartments in the Quanti-Tray example above.

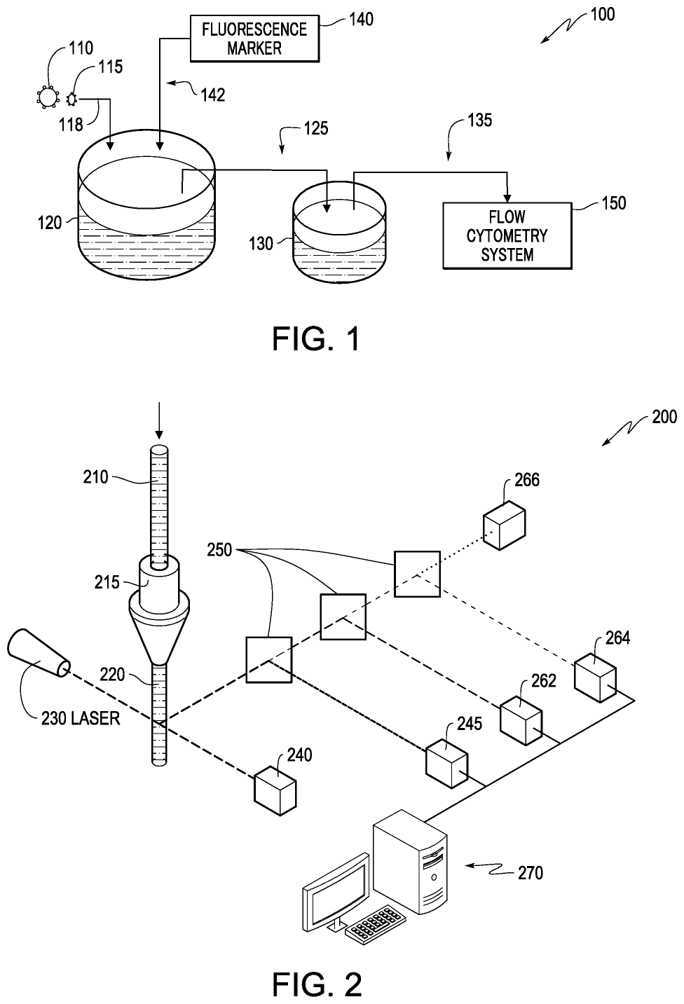

Referring to FIG. 1, functionalized beads 110, 115 having two different sizes are combined in step 118 with a sample 120 that includes the target of interest. The sample can be a liquid sample that includes any type of liquid. In one embodiment, the sample can be water (e.g., at least 95 wt. % water or at least 99 wt. % water), such as municipal drinking water, wastewater, industrial wastewater, industrial cooling water, boiler water, environmental and natural waters, beverages, bottled water, etc. The sample 120 to which the beads are added can have a volume of, for example, from 10 ml to 1,000 L, from 100 ml to 100 L, from 250 ml L ml to 10 L, or from 500 mL to 5 L. In some applications, such as municipal water testing, a minimum or target sample size may be set by regulation. The target(s) of interest in the sample can include a population of cells, such as specific microorganisms (e.g., conforms including E. coli, Legionella, Pseudomonas, Enterococcus, Giardia, Cryptosporidium, etc.). The target can also include live ones of the microorganisms and/or dead ones of the microorganisms. Alternatively, the target(s) can include particles or chemical compounds of interest in the sample. The target component can be present in the sample in concentrations ranging, for example, from 0 to 1000 per 100 mL, from 1 to 500 per 100 mL, from 10 to 250 per 100 mL, or from 30 to 100 per 100 mL.

The beads 110, 115 can be magnetic, which helps enable their separation from sample 120, as explained further below. The beads can be made from iron oxide or other magnetic or paramagnetic material. The beads 110, 115 can be surface functionalized by known techniques with any group or moiety that will capture or bind to the target cells, particles, or chemical compounds of interest. For example, the beads can be functionalized with antibodies, antibody conjugates, aptamers, DNAzmes, molecular imprinted polymers, heme groups, etc. The beads may also be functionalized with more than one group or moiety so that more than one target can be simultaneously detected. Generally, the beads 110 and 115 that are added to sample 120 can be surface-functionalized with the same groups or moieties so that the beads 110, 115 in each group can bind to the same target(s) in sample 120. The beads may be generally spherical, but the term “bead” as used herein is not limited to any particular shape. For example, the beads can be spheroidal, ovoidal, ellipsoidal, oblong, platelet-shaped, etc.

In general, the beads 110, 115 can be any size that is detectable by a flow cytometer, and the particular sizes chosen may depend on the capabilities of the flow cytometer. For example, the beads can have a dimension, e.g., diameter, in the range of from 25 nm to 75 microns, from 100 nm to 25 microns, from 200 nm to 10 microns, or from 500 nm to 1 micron. In FIG. 1, the beads 110, 115 that are added to sample 120 are illustrated as having two discrete sizes, but more than two size groups can be used. For example, beads with from 2 to 20 different sizes may be used, such as from 3 to 10, or 4 to 7 size groups. In some aspects, each size group can have a bead dimension, e.g., diameter, that is at least 20% different (larger or smaller) than the bead dimension of the next closest size group, and preferably at least 25% different. The beads 110, 115 can be provided in a suspension that includes known concentrations of each size group, and a known quantity of the suspension can be added to sample 120 in step 118. Alternatively, each size group can be provided as a separate suspension with a known concentration, and a known quantity of each suspension can be added to sample 120 in step 118. A number of beads in each size group that is added to sample 120 can vary based on the application, e.g., from 10 to 10,000, from 50 to 5,000, or from 100 to 1,000, for example.

Once the beads 110, 115 are combined with sample 120, the functional groups on the beads are allowed to bind to the target cells, particles, and/or chemical compounds in the sample 120 for a time period. The sample 120 can be mixed or agitated during this time period. The target(s) bind or adhere to the different sized beads.

In step 142, at least one fluorescence or colorimetric marker 140 is combined with sample 120 to label the target. In this regard, the marker 140 binds to or reacts with the target of interest, which allows the flow cytometer to detect fluorescence excitation, emission, and/or light absorbance and thus the presence of the target on a bead. In microbiological assays where a live/dead count is important, any of a number of vital dyes can be used as the marker 140 depending on the target organism, including, e.g., fluorescein diacetate, erythrosin B, SYTO9/propidium Iodide, 5-(And 6-)-carboxyfluorescein diacetate, succinimide ester, cyanoditolyl tetrazolium chloride, etc. If live/dead discrimination is not important, the marker 140 can be selected so that a sandwich-type assay (e.g., ELISA) that employs antibodies or other cellular recognition methods can be used. The marker 140 may be combined with sample 120 before the beads 110, 115 are added or at the same time the beads are added to react with the target of interest, or alternatively can be combined with sample 120 after that beads 110, 115 are added to react with the targets that are already attached to the beads. The marker 140 can alternatively be added to reduced-volume sample 130 to bind to the targets of interest.

In step 125, the beads 110, 115 are separated from the sample 120 to provide a reduced-volume sample 130 that includes a higher concentration of beads 110, 115. At this stage, the target(s) in the fluid sample are bound to at least some of the beads. Where the beads 110, 115 are magnetic, they can be magnetically separated from sample 120. Once separated, the beads can be rinsed to remove any extraneous material. As indicated above, the fluorescence marker 140 can optionally be added after the separation step to tag or label the targets.

At step 135, the reduced-volume sample 130 with the beads 110, 115 and targets can be run through a flow cytometer.

The processes described above can be adapted for use with any flow cytometer. One flow cytometer is illustrated schematically in connection with FIG. 2. In FIG. 2, flow cytometer 200 can include sample sheath 210, nozzle 215, flow sheath 220, laser 230, forward scatter detector 240, side scatter detector 245, fluorescent detectors 262, 264, 262, optical filters 250, and processing system 270. To detect the target(s) of interest in the fluid, the reduced-volume sample 130 is injected into sample sheath 210 and nozzle 215 generally distributes or arranges the beads 110, 115 in single file in the flow sheath 220. The laser 230 passes light through the flow sheath 220 and hits the beads as they flow through the flow sheath 220. Light scatter is detected by the forward scatter detector 240 and side scatter detector 245. The size and granularity of the beads/targets can be detected by the scatter detectors 240, 245. The optical filters 250 may include dichroic mirrors and direct particular bands of wavelength of light to each of the detectors. Fluorescence intensity at a particular band of wavelengths is detected by the fluorescence detectors 262, 264, 266. The fluorescence emission of a particular marker 140 can identify the presence of a specific target on a bead. Optionally, the flow cytometer can include a colorimetric detector that measures light absorbance over a range of wavelengths. The detectors 240, 245, 262, 264, and 266 or associated electronics (e.g., amplifier, A/D converter) can convert the detected light signals into electronic digital signals that can be processed and stored in processing system 270, for example. The processing system 270 can include a memory, processor (e.g., CPU), display, and user interface.

The processor can tag each bead that passes through the flow cytometer as either yes or no for the targets of interest, and can classify the targets into bins based on the detected size of the beads. The processor can also determine the MPN number of each target in sample 120 based on the detected signals. Similarly to the Quanti-Tray example described above, the MPN number of each target in the sample 120 can be extrapolated based on the number of yes hits in each size bin by using one or more statistical tables and/or a statistical MPN calculation, As indicated above, a live/dead count can also be determined based on the yes hits for certain dyes.

MPN calculations are known in the art, and a suitable MPN formula can be similar to those used for the Quanti-Tray assay where, e.g., constants representing the surface area or diameter of each bead size can be used in place of the compartment volume. Likewise, statistical tables can be generated that correlate the number of yes hits for each bead size group with the MPN of the target of interest.

Since the methods described herein can be used on any flow cytometer, one aspect of this invention is a kit that includes surface-functionalized beads having at least two discrete sizes and at least one fluorescence marker. The surface of the beads can be functionalized with groups or moieties that will bind with a specific target (e.g., specific cell, microorganism, particle, or chemical species), and the fluorescence marker can be selected to react with the target to provide a measurable fluorescence signal at a given wavelength. The beads can be provided in single suspension that includes all size groups, or alternatively, each bead size group can be provided in a separate suspension. The beads are present in the suspensions in predetermined concentrations. The kit can optionally include the statistical tables that allow a user to quickly identify the MPN of the target based on the number of yes hits in each size group. The components of the kit can be packaged in any suitable container.

It will be appreciated that the above-disclosed features and functions, or alternatives thereof, may be desirably combined into different systems or methods. Also, various alternatives, modifications, variations or improvements may be subsequently made by those skilled in the art. As such, various changes may be made without departing from the spirit and scope of this disclosure.

What is claimed is:

1. A method of analyzing a fluid sample to determine an amount of a target component in the fluid sample, the method comprising:
combining a first group of beads with the fluid sample, the first group of beads having beads with a first size that include a surface functional group and/or moiety that can bind to the target component;
combining a second group of beads with the fluid sample, the second group of beads having beads with a second size that is different from the first size and that include the surface functional group and/or moiety that can bind to the target component;
allowing the target component to bind to the surface functional group and/or moiety on the beads of the first and second groups of beads;
labeling the target component with a fluorescence marker;
separating the beads of the first and second group of beads from the fluid sample and then analyzing the separated beads with a flow cytometer to determine (i) a number of beads in the first group of beads that includes the target component; and (ii) a number of beads in the second group of beads that includes the target component.

2. The method of claim 1, further comprising determining a most probable number (MPN) of the target component in the fluid sample based on (i) the determined number of beads in the first group of beads that includes the target component; and (ii) the determined number of beads in the second group of beads that includes the target component.

3. The method of claim 1, wherein the beads are magnetic beads, and the step of separating includes magnetically separating the first and second group of beads from the fluid sample.

4. The method of claim 1, wherein the step of analyzing comprising detecting fluorescence emission of the fluorescence marker.

5. The method of claim 1, wherein the fluid sample includes water, and the target component is a microorganism.

6. The method of claim 5, wherein the fluorescence marker includes a dye that differentiates live ones of the microorganism from dead ones of the microorganism.

7. The method of claim 6, further comprising determining the most probable number (MPN) of the live microorganisms in the fluid sample and the most probable number (MPN) of the dead microorganisms in the fluid sample.

8. A method of analyzing a fluid that includes a target component, the method comprising:
combining at least two bead groups with the fluid, wherein each of the bead groups includes a plurality of surface-functionalized beads that have a different size from beads in each of the other bead groups, and wherein the target component in the fluid binds to at least some of the plurality of surface-functionalized beads;
binding a fluorescent or colorimetric marker to the target component;
then introducing the beads of the at least two bead groups into a flow cytometer, and detecting, with the flow cytometer, a size of each of the beads and fluorescence or colorimetric properties of each of the beads.

9. The method of claim 8 further comprising determining, based on the detected size and fluorescence or colorimetric properties of the beads, a number of beads in each of the at least two bead groups that includes the target component.

10. The method of claim 9, further comprising determining the most probable number (MPN) of the target component in the fluid based on the determined number of beads in each of the at least two bead groups that includes the target component.

11. The method of claim 8, wherein the at least two bead groups includes 2 to 20 bead groups.

12. The method of claim 8, wherein the at least two bead groups includes 3 to 10 bead groups.

13. The method of claim 8, further comprising separating the beads of the at least two bead groups from the fluid sample prior to the step of introducing the beads into the flow cytometer.

14. A kit comprising:
a first group of magnetic beads having a first size;
a second group of magnetic beads having a second size that is different from the first size; and
a fluorescence marker;
wherein the first and second groups of magnetic beads are surface-functionalized with the same functional group or moiety.

15. The kit according to claim 14, wherein the first and second groups of magnetic beads are provided as a suspension.

16. The kit according to claim 14, wherein the beads in the first and second groups of magnetic beads each have a diameter that is in a range of from 25 nm to 75 microns.
